(19) United States
(12) Patent Application Publication
(10) Pub. No.: US 2022/0110715 A1
(43) Pub. Date: Apr. 14, 2022

(54) Film Tensioning Element

(71) Applicant: Ivoclar Vivadent AG, Schaan (LI)

(72) Inventors: Markus Lichtensteiger, Montlingen (CH); Walter Pokorny, Feldkirch (AT); Frank Müller, Feldkirch (AT)

(21) Appl. No.: 17/474,106

(22) Filed: Sep. 14, 2021

(30) Foreign Application Priority Data
Oct. 9, 2020 (EP) 20200962.7

(51) Int. Cl. A61C 5/90

(52) U.S. Cl. CPC A61C 5/90

(57) ABSTRACT

The invention relates to a film tensioning element (10), in particular for performing dental operations in a patient's oral cavity, comprising a vestibular ring (16) and a lip ring (14) between which and optionally beyond which a film (12) extends, in particular substantially in a ring shape or hose shape, said film being held tensioned by the rings (14, 16), the film (12) and the rings (14, 16) being resiliently deformable. The film (12) has a radially inwardly extending constriction (18), at least in a region, in the progression between the lip ring (14) and vestibular ring (16), the film (12), at least in said region, extending without tension and in a single piece between the radially inwardly directed inner face (36) and radially outwardly directed outer face (38) thereof, in particular without overlays and/or inlays.

### CROSS-REFERENCE TO RELATED APPLICATIONS

This application claims priority to European patent application No. 20200962.7 filed on Oct. 9, 2020, which disclosure is incorporated herein by reference in its entirety.

### TECHNICAL FIELD

The invention relates to a film tensioning element.

### BACKGROUND

Film tensioning elements have been successfully in use for more than ten years, under the name OPTRAGATE®, for allowing free access to a patient's mouth during dental operations in the patient's oral cavity. It consists of a vestibular ring and a lip ring between which a film extends. In this element, the film is mounted displaceably with respect to the two rings and is resilient. To avoid allergy-related damage, it is formed latex-free.

US 20040097795 and US 20080153058 are directed to lip and cheek expanders and are hereby incorporated by reference in their entirety. Further, EP 3 666 221 A1, US 20200188060A1 and US 20210177251A1, which published US patent applications are hereby incorporated by reference, disclose a film tensioning element in which an elastic band is integrated into the film. The elastic band doubles up the film, in such a way that this combination of the film and the tensioning element has a greater tensile force there than the film otherwise would have alone. The radially inwardly directed tension on the film from the elastic band results in a constriction, both of the elastic band and of the film, in such a way that the radially outwardly directed pressure on the lips is reduced.

This embodiment has the drawback, by comparison with the other embodiments disclosed in said document, that it is comparatively complex and expensive to manufacture. In addition, the turned-over elastic band causes a permanent tension within the film and the band.

During the storage period, this constriction effect can slacken and lose its effect.

It would be possible to integrate the elastic band into the film, for example by peripheral sealing. However, the radially inwardly directed tensile force leads to fold formation, and as a result, one of the other embodiments described in said patent publication has become established instead of this solution using the doubled-up elastic band.

### SUMMARY OF THE INVENTION

By contrast, the object of the invention is to provide a film tensioning element in accordance with claim 1 which improves the comfort of wearing the film tensioning element.

This object is achieved according to the independent claims. Advantageous embodiments may be drawn from the dependent claims.

Surprisingly, the combination of the measures according to the invention, in other words both providing the film itself with a radially inwardly extending constriction and also manufacturing the film without tension, in other words with the constriction arising in the film by itself without tension, without a tensile force having to be implemented using an elastic band which thickens the film, leads to the advantage that no folds occur, but yet no radially outwardly directed pressure is exerted on the lips.

Surprisingly, a further advantage also occurred, specifically that it is possible to save on material. The film may have a constant material thickness over the progression thereof, and this results in saving on material, both by comparison with the presented embodiment in FIG. 6 of EP 3 666 221 A1, US 20200188060A1 and US 20210177251A1, and even with respect to the conventional OPTRAGATE® film tensioning solution where the film extends between the vestibular ring and the lip ring in a frustum shape, if the film tensioning element is held on one of the rings.

The film tensioning element is a mass-produced product. Considered on an industrial scale, this material saving affects the weight and allows a considerable reduction in manufacturing costs.

The film extends between the lip ring and vestibular ring in a ring or hose shape in a manner known per se.

An advantageous configuration provides that the constriction is merely provided over part of the periphery. As is known, a half-open mouth has a substantially oval shape. This shape can now be taken into account in that the constriction is central, in other words extends for example over an upper and a lower quarter-circle when the film tensioning element is inserted into the mouth, and no constriction is provided in the region of the two outer quarter-circles. As a result, a particularly scanning-friendly configuration is implemented, which also makes it possible to scan side regions of the patient's mouth.

The material thickness of the film is preferably constant over the entire film.

The material thickness may for example be between 0.1 mm and 1 mm, and is preferably between 0.3 and 0.4 mm. The material thickness of the film of a film tensioning element provided to children is preferably slightly less, for example 0.2 or 0.3 mm. This reduces the tensile force of the film, and surprisingly no folds occur either, or at most very slight folds, in a smaller configuration of this type of the film tensioning element according to the invention.

According to the invention, it is also particularly favorable that the point at which the constriction is implemented can be adapted to requirements within wide ranges. In a preferred configuration, the constriction extends more closely adjacent to the vestibular ring than to the lip ring. As a result, it is possible to configure the vestibular ring with a comparatively low diameter, and this in turn leads to a savings on material. This reduction in diameter may for example be between 10 and 20 percent.

In another configuration, the constriction is provided close to the lip ring.

Because the film is free of overlays and/or inlays, the tensile force when loaded is constant over the entire extension thereof. This applies both in the longitudinal direction, in other words the direction between the vestibular ring and the lip ring, and in the tangential direction, in other words peripherally.

This also results in introduced tensions being absorbed uniformly over the entire extension. As a result, the film is loaded uniformly by the introduced radially inwardly acting tension. The load per unit area is thus much less than when an additional elastic band is intensively connected to the film.

The constant thickness which is provided in an advantageous configuration thus has the particular advantage that the tear resistance is increased by comparison with the described previously known solution. This measure again makes it possible to reduce the production costs, since the solution using the thickening is less tear-resistant than the solution according to the invention, in spite of having the same size for the same material weight.

Although a fluctuation of for example 10 percent in the material thickness does not affect the weight as regards to the stability of the film, a significant thickening, formed as an elastic band and extending over for example a third of the length of the film in the longitudinal direction, does weaken the remaining part of the film; the stretching of the film occurs almost exclusively in the remaining two thirds of the film, at least if the material thickness of the thickening is for example three times the film thickness elsewhere, for example as a result of the integrated elastic inlay.

Thus, in spite of a higher total weight, the tear resistance of the film tensioning element of the previously known solution is only approximately two thirds of the tear resistance of the film tensioning element according to the invention.

The depth of the constriction can be adapted to requirements within wide ranges. Thus, the depth may also equally well have an oval progression. Thus, in a first configuration it is largest in the mouth center region and smallest in the side region.

In a second configuration, these relationships are reversed, the depth being largest in the side region and smallest in the mouth region.

It is also possible to provide the depth of the constriction to be variable, as considered in a tangential direction, from the outset, with soft transitions between the different depths, and, when the film tensioning element is used on and in the patient's mouth, to rotate the film tensioning element in such a way that the comfort of wearing is thus optimal. As a result of the different shapes of patients' mouths, optimized patient-specific adaptation can thus be achieved using this measure.

The film tensioning element may be manufactured in any desired manner. For example, the film may be produced by an injection-molding process, the injection mold predetermining the configuration of the subsequent shape of the film in the limp state. The rings may be inlaid in the injection mold and be molded around.

A dipping process or blow-molding process is also possible instead.

It is also possible to provide the film with variable thickness as considered about the periphery thereof in particular cases, the differences, in this case too, preferably not being more than 20 percent, and soft transitions again being provided between the different thicknesses of the films.

For the above-described particularly good tear resistance of the film, it is important that the film has a constant thickness as considered in the longitudinal direction. The greatest introduced tension comes from a patient's lip, which tensions the film in the longitudinal direction.

If the thickness progression is to be selected to be variable in the longitudinal direction, it is preferred to limit the fluctuation and the differences to ±20 percent in this case too.

In a further advantageous configuration, a net structure of the film is provided using the finite element method (FEM). This is particularly preferably implemented in such a way that the meshes of the net have a lower film wall thickness than the lattice of the net. This does not impair the sealing by the film according to the invention, but the strength in relation to the weight used is further improved.

This applies in particular if the net structure is determined while taking into account a calculation by the finite element method.

The radial depth of the constriction can be adapted to the requirements within wide ranges. It is dimensioned as a reduction in diameter with respect to a standard OPTRAGATE® film tensioning element, in other words the prior art film tensioning element.

Said element extends in a frustum shape, at least when it is held suspended on the lip ring or vestibular ring without an additional tension and/or tensile forces being introduced.

When the film tensioning element according to the invention is held suspended on the lip ring or vestibular ring without an additional tension and/or tensile forces being introduced, it has a shape having a radially inwardly extending constriction.

The depth of the constriction may be 5 to 35% of the diameter of the lip ring, in particular approximately 15%.

According to the invention, it is particularly favorable for the film to extend in one piece. The single-piece nature applies in all directions, in other words in a direction parallel to the axis between the vestibular ring and lip ring, in a tangential direction, and also in the thickness direction thereof.

This means that the film is likewise a single-piece between the radially inwardly directed inner face thereof and the radially outwardly directed outer face thereof, and has no overlays or supports and/or inlays or inserts.

As a result of the single-piece nature, a tension gradient within the film is prevented or reduced. This increases the tear resistance, and tension peaks do not occur anywhere, but instead the introduced tensions are made uniform.

The constriction according to the invention may also be considered as a concavity or inward curvature. What is important is the change in diameter as considered along the axis. Starting from the lip ring, the diameter initially decreases, specifically without points of discontinuity and initially decreasing monotonically.

In the region of the deepest constriction, the diameter of the film is lowest. There is an inflection point here, but again no point of discontinuity. Starting from this inflection point, the diameter of the film increases again until the vestibular ring.

Even though other curve shapes of the film are possible in this regard, implementation of a catenary or a rounded-tip V is preferred.

It is preferred that a film tensioning element for performing dental operations in a patient's oral cavity is provided having a vestibular ring and a lip ring between which vestibular ring and lip ring, and optionally beyond which, a film extends, the film being held tensioned by the rings, the film and the rings being resiliently deformable, wherein the film has a radially inwardly extending constriction, at least in a region between the lip ring and vestibular ring, the film at least in said region extending without tension and in a single piece between an inner face and an outer face thereof free of any supports and/or inserts.

It is preferred that the film has, in the region between the lip ring and vestibular ring, a constant thickness within a standard of deviation of ±20% of the constant thickness, and/or wherein the film has a lower diameter at the constriction than at the rings.

It is preferred that the region of the constriction is ring-shaped (360 degrees) or part-ring-shaped or partially ring-shaped (less than 360 degrees).

It is preferred that the region of the constriction extends over less than 99% and more than 30% of a distance between the vestibular ring and the lip ring. Depending on how the film tensioning element is manufactured, it has a “broader” or “less broad” constriction—in the side view. “Broad” would be 99%, reaching from the vestibular ring to the lip ring. If the constriction extends only over a third of the distance between the rings it would be “less broad”.

It is preferred that the depth of the constriction, as considered radially inwards, is variable over the periphery of the film.

It is preferred that the depth of the constriction is greater at a point intended for the cheeks. “Greater” can mean that it is greater compared to the tangential remainder of the depth. The depth may have a range of for example, 1% to 30%, preferably between 10% and 20%.

It is preferred that the depth of the constriction is greater at a point intended for the mouth center at and around the sagittal plane.

It is preferred that the constriction shapes the film radially inwards without tension and/or a point of the greatest depth of the constriction is in the center between the rings or shifted towards the lip ring with respect to the center, the center being half way from the lip and vestibular rings.

It is preferred that the film, as considered in a section through the lip ring and vestibular ring, extends in a catenary manner in the limp state, or wherein the film, as considered in a longitudinal section parallel to the axis of the film tensioning element, extends substantially in a manner of a V or is V-shaped.

It is preferred that the film is manufactured by an injection-molding process, a pressing process, a dipping process or a blow-molding process.

It is preferred that the film has different thicknesses about the periphery of the film tensioning element, between the lip ring and vestibular ring.

It is preferred that the constriction of the film has a maximum depth of at least 5 times or at least 10 times or at least 15 times the maximum thickness of the film.

It is preferred that the constriction the film has different wall thicknesses in the periphery.

It is preferred that the film has a net structure, which is established while taking into account an FEM calculation. It is preferred that the meshes of the net have a lower wall thickness than the lattice of the net.

### BRIEF DESCRIPTION OF THE DRAWINGS

Further advantages, details and features of the invention may be drawn from the following description of a plurality of embodiments with reference to the accompanying drawings, in which:

FIG. 1A is a schematic three-dimensional line view of an embodiment of a film tensioning element according to the invention;

FIG. 1B is a plan view of an embodiment of a film tensioning element according to the invention;

FIG. 1C is a plan view of an embodiment of a film tensioning element according to the invention;

FIG. 1D is a plan view of an embodiment of a film tensioning element according to the invention;

FIG. 1E is a sectional view of an embodiment of a film tensioning element according to the invention;

FIG. 1F is a sectional view of an embodiment of a film tensioning element according to the invention;

FIG. 2A is a sectional view of an embodiment of a film tensioning element according to the invention;

FIG. 2B is a sectional view of an embodiment of a film tensioning element according to the invention;

FIG. 2C is a sectional view of an embodiment of a film tensioning element according to the invention; and

FIG. 3 is a side view of a further embodiment of a film tensioning element according to the invention.

### DETAILED DESCRIPTION

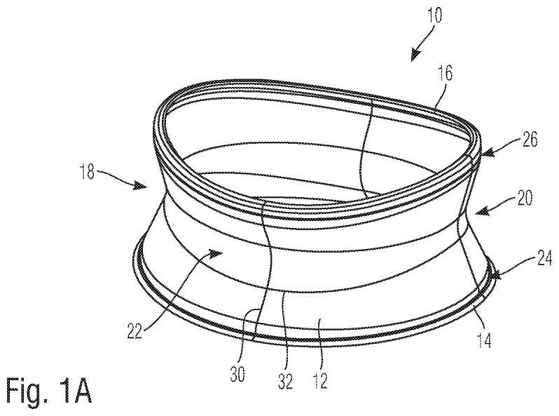

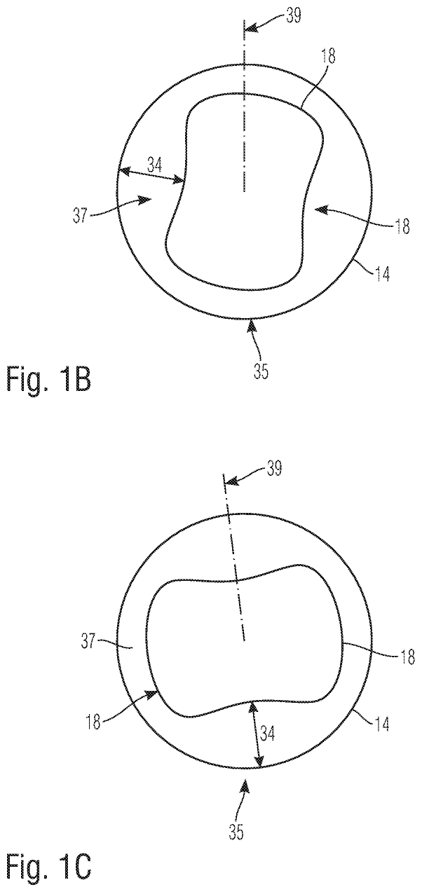

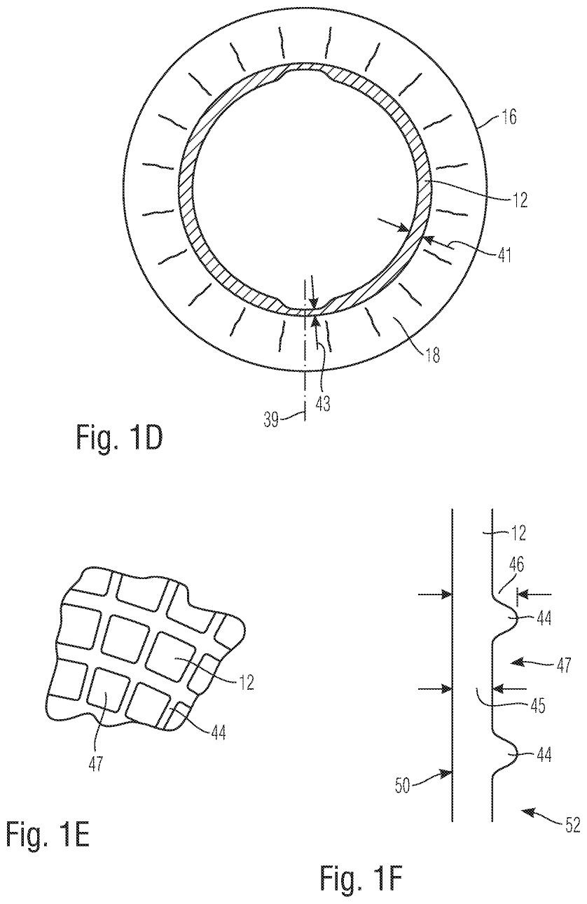

FIG. 1A shows a film tensioning element 10, which in a manner known per se consists of a film 12, a lip ring 14 and a vestibular ring 16. The film tensioning element 10 as a whole is ring-shaped, the basic construction substantially corresponding to a circular ring, but also being able, in a modified embodiment, to have a different ring shape, such as an oval or elliptical shape.

In the embodiment shown, in a manner likewise known per se, the vestibular ring 16 has a somewhat smaller diameter than the lip ring 14.

According to the invention, the film 12 is constricted between the two rings 14 and 16.

In the embodiment shown, this constriction 18 is formed symmetrically, in such a way that the deepest point of the constriction 18 is in the center between the lip ring 14 and vestibular ring 16.

In the embodiment shown, the constriction 18 is formed ring-shaped peripherally, in such a way that at all points of the ring-shaped film tensioning element 10 the smallest or lowest diameter of the film tensioning element is in the region of the constriction 18.

In the embodiment shown in FIG. 1A, the constriction 18 extends substantially in V shape or is in a V shape, such that the film is angled or slightly or somewhat angled from the deepest point of constriction outwardly toward each ring 14, 16. The deepest point of the V 20 of the constriction 18 is provided with a radius 22, which in the embodiment shown is less than the depth of the constriction 18.

The position and shape shown in FIG. 1A of the film tensioning element 10 occurs when the film tensioning element 10 is without tension. If for example the lip ring 14 is laid on an underlay and the film tensioning element 10 is raised on the vestibular ring 16 until the shape shown in FIG. 1A occurs, the film 12 is without tension (with the exception of the very low inherent weight thereof). In this shape, the film 12 is free or virtually free of folds and the constriction 18 is represented by a particular preformed shape of the film 12.

The film tensioning element 10 may be manufactured for example in an injection-molding process. Preferably, in this case the lip ring 14 and vestibular ring 16 are laid in the injection mold and are molded around with the material of the film 12. In a manner known per se, the material of the film 12 is resilient and comparatively soft, while the rings 14 and 16 consist of a comparatively harder plastics material.

The injection mold is formed in such a way that, starting from the rings 14 and 16, the thickness of the film 12 tapers continuously and in a soft transition. The region of the taper can be kept small, for example approximately corresponding to the thickness of each ring 14 or 16. This solution makes possible, with low use of material, a construction of the film 12 which is optimized in terms of load capacity.

In the depiction of FIG. 1A, a standard OptraGate® film tensioning element would have a conical construction without a constriction 18.

The load capacity of the film tensioning element 10 according to the invention is at least equally good. However, the material use is slightly less, for example by 10 to 20%, depending on the depth of the constriction 18. As a result of the reduction in diameter in the region of the constriction 18, less material is required.

By comparison with the likewise known film tensioning element 10 according to the above-mentioned patent publication, which has a doubled-up elastic band, the material use is much lower, since no elastic band is used, but rather the film 12 has a constant, uniform thickness over the extension thereof between the rings 14 and 16, again with the exception of the aforementioned taper 24.

Surprisingly, the centrally provided constriction 18, which is preferably V-shaped, results in an extremely wearer-friendly adaptation to the lips of the patient, which reduces the lip pressure and thus increases acceptance.

Surprisingly, by way of the central radius 22, a considerable improvement can thus be achieved by comparison with an asymmetrically arranged elastic band.

A major advantage of the invention is that, by way of appropriate tool configuration, for example the injection mold shape, the constriction may be positioned centrally, but also equally well non-centrally, and thus for example closer in part to one of the two rings or optionally with different V depths. It may also, as a whole, only be present in part of the tensioning element.

Since the thickness of the film 12 is uniform or substantially uniform over the entire extension thereof between the two rings 14 and 16, introduced tensions are distributed uniformly. The load capacity is thus likewise surprisingly improved according to the invention by comparison with the elastic band solution, in spite of the higher material use of said solution.

For clarity, it should be noted that the lines additionally shown in FIG. 1A, for example lines 30 and 32, are merely shape lines which are intended to indicate the shape of the film tensioning element 10 but are not present on the actual film tensioning element 10. Rather, the film 12 actually extends smoothly and without overlays and transitions between the rings 14 and 16, but also along the ring shape thereof.

FIG. 1B shows the depth 34 of constriction 18 as not being constant. Depth 34 is shown as greater at lateral regions near 37, intended for the cheeks and smaller at the center 35. Examples of the range in the depth 34 at region 37 may be from 1% to 30%, and preferably between 10% and 20% greater in depth at region 37 in comparison to the center region 35.

FIG. 1C shows the depth 34 of constriction 18 as not being constant. Depth 34 is shown as smaller at lateral regions near 37, intended for the cheeks and greater at the center 35. Examples of the range in depth 34 at center region 35 may be from 1% to 30%, and preferably between 10% and 20% greater in depth at the center region 35 in comparison to region 37.

FIG. 1D shows a sectional view of the film tensioning element with the film 12 parallel and close to the vestibular ring 16 having different thicknesses 41, 43 about the periphery of the film tensioning element, between the lip ring 14 and vestibular ring 16. The thickness 43 of the film 12 is smaller close to the sagittal plane 39 and greater close to the cheeks. Moreover, the constriction 18 is shown having different thicknesses in the periphery.

FIG. 1E shows a view of a small section of film 12. Film 12 may have a net structure with ribs 44 forming a grid and extending on one or both sides of film 12. The net structure is for reinforcing the film. The net distribution may be selected in an appropriate manner. The distribution may be based on an FEM analysis.

FIG. 1F shows a sectional view through such a net structured film 12. Ribs 44 forming a lattice have a greater thickness at 46 than thickness 45 of meshes 47. Yet thickness 45 is not zero such that film 12 has no through holes. In this embodiment, the left side 50 is flat and right side 52 is equipped with ribs 44.

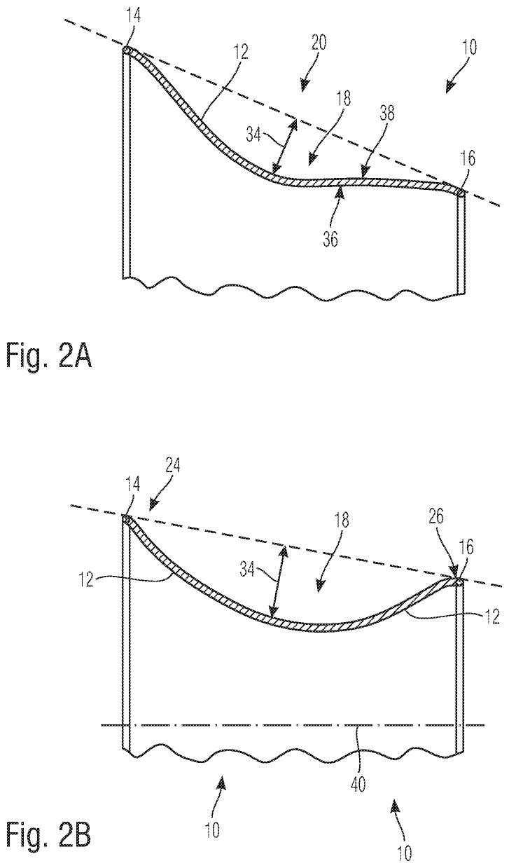

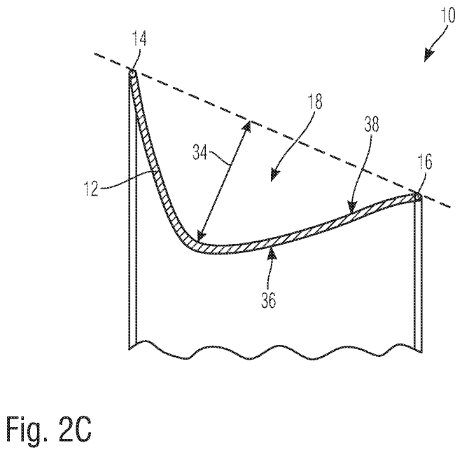

From FIG. 2A and FIG. 2B, two different shapes of film tensioning elements 10 can be seen in section.

FIG. 2A shows a film tensioning element 10 comprising a film 12, which extends with a constriction 18 substantially in the shape of a V 20 between the rings 14 and 16. In the embodiment shown, the deepest point of the V 20 is in the center between the rings 14 and 16. In the embodiment shown, the depth 34 of the constriction 18 is more than 15 times the thickness of the film 12. It may be less than 70, 60, 50, 40, 30 or 20 times the thickness of film 12. The “thickness” means the extension of the film 12 between the radially inwardly directed inner face 36 thereof and the radially outwardly directed outer face 38 thereof. Within this thickness, the film 12 extends homogeneously in a unitary material.

In the embodiment shown, the thickness of the film 12 is constant between the rings 14 and 16, optionally with the exception of the associated tapers 24 and 26. In the embodiment of FIG. 2A, however, no tapers 24 and 26 are provided; instead, in this embodiment, each ring 14 or 16 is flattened in such a way that the film 12 adjacent to the ring extends away from said ring with a substantially constant thickness.

By contrast, both in the embodiment of FIGS. 1A and 1n the embodiment of FIG. 2B, the thickness of the rings 14 and 16 is greater than the thickness of the film 12, and so tapers 24 and 26 are preferably provided.

In the embodiment shown in FIG. 2A, the thickness of the film 12 is 0.4 mm.

It will be appreciated that any other film thicknesses adapted to the size and shape of the film tensioning elements 10 are also possible, for example between 0.1 mm for small film tensioning elements for children's mouths and 1 mm in the event of a heavy load or in applications on larger mouths, in which film tensioning elements 10 of this type may likewise be used.

As can be seen from FIG. 2A, the length of the rings 14 and 16 along the extension of the film tensioning element 10 in the direction of the film 12 is increased with respect to the radial extension. This shape leads to increased stability against axial deformation, but also good adaptability to the mouth shape of the patient, in particular in the region of the vestibular ring 16.

A modified embodiment of the film tensioning element 10 according to the invention can be seen from FIG. 2B. In this embodiment, the film 12 extends with the constriction 18 in the manner of a catenary curved between the rings 14 and 16. The rings 14 and 16 have a circular construction or substantially circular construction in cross section and a thickness of 1.2 mm. The thickness of the film 12 is shown somewhat exaggerated, and in this embodiment is 0.3 mm. In this case too, the thickness is uniform and free of overlays and inlays over the entire extension of the film 12 between the rings 14 and 16.

In FIG. 2B, an imaginary axis 40 of the film tensioning element 10 is also drawn in. This is also present for the other embodiments of the film tensioning element 10, even though it is not drawn in there. It illustrates in which direction a radial extension or radial direction is conceived.

In the embodiment of FIG. 2B, the depth 34 of the constriction 18 is 30 times the thickness of the film 12.

Even though in this case the deepest point of the constriction 18 extends in the center between the rings 14 and 16 in each case, it will be appreciated that a slight deviation thereof either towards the vestibular ring 16 or towards the lip ring 14 is possible without departing from the scope of the invention.

In a modified embodiment, it is provided that the constriction 18 is larger at the point intended for the cheeks.

In an alternative embodiment, it is provided that the depth 34 of the constriction 18 is greater at the point intended for the mouth center. It is thus greatest in the region of the patient's sagittal plane.

Although in this case the constriction 18 is provided with a constant depth 34 over the entire ring-shaped progression of the film 12, in other words in the circumferential direction or tangential direction, in a further embodiment it is provided that the depth 34 of the constriction 18 is varied over the ring shape. The constriction 18 may also merely extend in a partial ring shape.

The region of the constriction 18 also need not extend over the entire distance between the vestibular ring 16 and lip ring 14; it is also possible to provide the constriction 18 for example centrally with a smaller extension parallel to the axis.

FIG. 2C shows a further embodiment of a film tensioning element 10 according to the invention. In this embodiment, on the one hand, the constriction 18 is considerably deeper. The depth 34 is more than half of the distance between the lip ring 14 and vestibular ring 16. In the embodiment of FIGS. 2A and 2B, by contrast, the depth 34 is only approximately 15% of the distance between the lip ring 14 and vestibular ring 16.

According to the invention, the depth 34 can be adapted to the requirements within wide ranges, for example by appropriate configuration of the injection mold. The depth 34 may, according to the invention, preferably be between 8% and 80% of the distance between the lip ring 14 and vestibular ring 16.

On the other hand, in the embodiment of FIG. 2C, the center of gravity of the constriction is considerably closer to the lip ring 14 than to the vestibular ring 16. The lip ring 14 is larger than the vestibular ring 16, because the diameter of the film 12 decreases considerably starting from the lip ring 14, for example to half of the diameter of the lip ring 14 within a quarter of the distance between the lip ring 14 and vestibular ring 16.

This considerable decrease in diameter saves a significant amount of film material.

In the embodiments shown in FIGS. 1A, 2A, 2B and 2C, the axial range of the constriction 18 is 99% for FIGS. 1A and 2B and 100% for FIGS. 2A and 2C.

As explained previously with reference to FIG. 1A, it is preferred for the film 12 to have the shape shown in the drawings without tension. It is thus in effect shaped or pre-shaped directed radially inwards without tension as a result of the constriction 18.

As an alternative to the injection molding process, it is also possible to manufacture the film tensioning element 10 according to the invention by a pressing process, a dipping process or a glass shaping process.

If the film according to the invention is manufactured from silicone materials, the rings may be laid in the press mold beforehand and encapsulated.

The film 12 may also have different thicknesses as considered over the periphery.

These different thicknesses correspond to the different depths 34 of the constriction 18.

In a direction parallel to the axis 40, however, the thickness of the film 12 is preferably always a constant thickness between the lip ring 14 and vestibular ring 16, optionally with the exception of tapers 24 and 26.

In a further modified configuration, it is provided that the film 12 has a net structure. This is preferably calculated by the finite element method, and serves to optimize the material saving further. The thickness of the film 12 is then for example merely 0.1 mm in the region of the meshes of the net and 0.3 mm in the region of the lattice of the net.

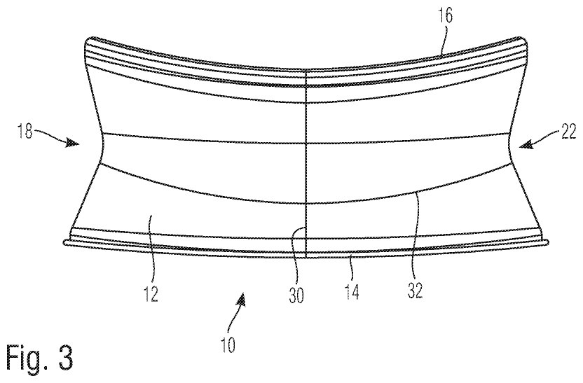

FIG. 3 is a different view of an embodiment of the film tensioning element 10 which is slightly modified with respect to the embodiment of FIG. 1A. As in the other drawings, like reference numerals indicate like or corresponding parts here.

In the embodiment of FIG. 3, the vestibular ring 16 and lip ring 14 can be curved in the side view shown, the vestibular ring 16 somewhat more so than the lip ring 14.

In this embodiment, tapers 26 and 24 may be provided in accordance with FIG. 1A. The rings 14 and 16 are molded around with the material of the film 12.

The term “about” and “substantially” is intended to include the degree of error or uncertainty associated with measurement of the particular quantity or shape as one of ordinary skill in the art would understand. For example, but not limited to, “about” and “substantially” can include a range of ±0.01% to 15%, or 2% to 10%, or 5% to 25% of a given shape or value.

As a result of the curved shape, the vestibular ring 16 can more easily be introduced into the patient's mouth, and also more easily takes on the desired oval shape, which corresponds to the patient's mouth.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. A film tensioning element for performing dental operations in a patient's oral cavity, comprising
a vestibular ring (16) and
a lip ring (14) between which vestibular ring and lip ring, and optionally beyond which, a film (12) extends, said film being held tensioned by the rings (14, 16), the film and the rings being resiliently deformable,
wherein the film (12) has a radially inwardly extending constriction (18), at least in a region, between the lip ring (14) and vestibular ring (16), the film (12), at least in said region, extending without tension and in a single piece between an inner face (36) and an outer face (38) thereof free of any supports and/or inserts.

2. The film tensioning element according to claim 1,
wherein the film (12) has, in the region between the lip ring (14) and vestibular ring (16), a constant thickness within a standard of deviation of ±20% of the constant thickness, and/or
wherein the film (12) has a lower diameter at the constriction (18) than at the rings (14, 16).

3. The film tensioning element according to claim 1,
wherein the region of the constriction (18) is ring-shaped, 360 degrees or part-ring-shaped or partially ring-shaped, less than 360 degrees.

4. The film tensioning element according to claim 1,
wherein the region of the constriction (18) extends over less than 99% and more than 30% of a distance between the vestibular ring (16) and the lip ring (14).

5. The film tensioning element according to claim 1,
wherein a depth (34) of the constriction (18), as considered radially inwards, is variable over the periphery of the film (12).

6. The film tensioning element according to claim 5,
wherein the depth (34) of the constriction (18) is greater at a point intended for the cheeks.

7. The film tensioning element according to claim 5,
wherein the depth (34) of the constriction (18) is greater at a point intended for the mouth center at and around the sagittal plane.

8. The film tensioning element according to claim 1,
wherein the constriction (18) shapes the film (12) radially inwards without tension and/or a point of the greatest depth (34) of the constriction is in the center between the rings (14, 16) or shifted towards the lip ring (14) with respect to the center, the center being half way from the lip and vestibular rings.

9. The film tensioning element according to claim 1,
wherein the film (12), as considered in a section through the lip ring (14) and vestibular ring (16), extends in a catenary manner in the limp state, or wherein the film (12), as considered in a longitudinal section parallel to an axis (40) of the film tensioning element (10), extends substantially in a manner of a V (20).

10. The film tensioning element according to claim 1,
wherein the film (12) is manufactured by an injection-molding process, a pressing process, a dipping process or a blow-molding process.

11. The film tensioning element according to claim 1,
wherein the film (12) has different thicknesses about the periphery of the film tensioning element, between the lip ring (14) and vestibular ring (16).

12. The film tensioning element according to claim 1,
wherein the constriction (18) of the film (12) has a maximum depth (34) of at least 5 times or at least 10 times or at least 15 times the maximum thickness of the film (12).

13. The film tensioning element according to claim 1,
wherein at the constriction (18) the film (12) has different wall thicknesses in the periphery.

14. The film tensioning element according to claim 1,
wherein the film (12) has a net structure, which is established while taking into account an FEM calculation.

15. The film tensioning element according to claim 14,
wherein the meshes of the net have a lower wall thickness than the lattice of the net.
